(19) United States
(12) Patent Application Publication
(10) Pub. No.: US 2014/0037798 A1
(43) Pub. Date: Feb. 6, 2014

(54) LIQUID NUTRITIONAL PRODUCTS COMPRISING METAL AMINO ACID CHELATES

(71) Applicants: Paul W. Johns, Columbus, OH (US); John W. McEwen, Columbus, OH (US); Gaurav Patel, Gahanna, OH (US)

(72) Inventors: Paul W. Johns, Columbus, OH (US); John W. McEwen, Columbus, OH (US); Gaurav Patel, Gahanna, OH (US)

(73) Assignee: ABBOTT LABORATORIES, ABBOTT PARK, IL (US)

(21) Appl. No.: 13/984,056

(22) PCT Filed: Feb. 8, 2012
(86) PCT No.: PCT/US12/24286

§ 371 Date: Oct. 9, 2013

Related U.S. Application Data
(60) Provisional application No. 61/441,092, filed on Feb. 9, 2011.

(51) Int. Cl. A23L 1/29

(52) U.S. Cl. CPC A23L 1/296
USPC 426/73; 426/72

(57) ABSTRACT

Disclosed are shelf stable nutritional liquids including metal amino acid chelates. Metal amino acid chelates, such as copper amino acid chelates and iron amino acid chelates, limit the capacity of incompletely bound copper and iron to catalyze the oxidation of sensitive nutrients such as vitamins, particularly vitamin A, vitamin B12 and vitamin C, thereby imparting both nutritional benefits and sensory benefits to the nutritional liquids.

### FIELD OF THE DISCLOSURE

The present disclosure relates to oxidatively stable nutritional liquids comprising metal amino acid chelates.

### BACKGROUND OF THE DISCLOSURE

Nutritional liquids comprising a targeted selection of nutritional ingredients generally including protein, lipids, vitamins and minerals are known and widely available. A common problem in some of these nutritional liquids is the oxidation of sensitive nutrient ingredients, and particularly the oxidation of many vitamins. This oxidation of vitamins may be metal-catalyzed oxidation. More particularly, incompletely bound soluble metals, such as iron and copper, within the nutritional liquid may generate reactive oxygen species (ROS), such as hydroxyl radicals, that can oxidize the vitamins and lead to losses of vitamin A, vitamin B12, and vitamin C. This loss can be enhanced when hydrolyzed proteins are used as the protein source, as these proteins can, in some cases, lead to increased nutrient oxidation.

These nutrient oxidation problems have previously been addressed mainly by over-fortifying liquid nutritional products with vitamins. Over-fortification of vitamin C however, is generally controlled. Other solutions attempted for controlling oxidation of sensitive nutrients in liquid nutritionals have included minimizing oxidation by reduced heat treating of the liquid nutritional; adjusting the order of addition of the ingredients to the nutritional liquids; and decreasing the pH of the nutritional liquids. To date, however, these solutions have not been able to completely inhibit the nutrient oxidation problems in some nutritional liquids.

Additionally, some approaches have been used to limit the catalytic activity of metals such as iron and copper in the liquid nutritional. These additional approaches, however, have generally led to a reduced bioavailability of iron and copper in the nutritional liquid.

It would therefore be desirable to formulate nutritional liquids with vitamins, and particularly vitamins A, B12, and C, such that the vitamins are more oxidatively stable over time, even in the presence of iron and copper. Additionally, it would be beneficial if the nutritional liquids could be formulated as stable liquids having an improved bioavailability of iron and copper, and could be formulated with hydrolyzed proteins.

### SUMMARY OF THE DISCLOSURE

One embodiment of the present disclosure is directed to a nutritional liquid comprising from about 2 mg per liter to about 3000 mg per liter metal amino acid chelate and at least one vitamin.

Another embodiment is directed to a nutritional liquid comprising a protein hydrolysate, a metal amino acid chelate, and at least one vitamin.

Another embodiment is directed to a nutritional liquid comprising from about 2 mg per liter to about 400 mg per liter copper amino acid chelate, at least one of vitamin B12 or vitamin C, and at least one nutrient selected from the group consisting of fat, protein, or carbohydrate.

It has now been discovered that metal amino acid chelates, and particularly, iron amino acid chelates and copper amino acid chelates, can reduce the catalytic activity of metals in nutritional liquids and thus reduce the oxidation and decomposition of oxidatively sensitive nutrients such as vitamin B12 and vitamin C. Additionally, by inhibiting oxidation of various nutrients, the inclusion of metal amino acid chelates in the nutritional liquid products may prolong the shelf life, as well as improve flavor and color in the nutritional liquid products.

Further, it has been found that the use of the iron amino acid chelates and/or copper amino acid chelates in the nutritional liquids may increase the bioavailability of the iron and/or copper as the iron and copper amino acid chelates are highly bioavailable species such that decreased concentrations of these ingredients can be utilized while maintaining sufficient bioavailability. Moreover, it has been found that the nutrient oxidation and bioavailability benefits are maintained even when the nutritional liquids include hydrolyzed protein.

### DETAILED DESCRIPTION OF THE DISCLOSURE

The nutritional liquids of the present disclosure comprise metal amino acid chelates to reduce or eliminate the catalytic oxidative activity of metallic species such as iron and copper in the liquids without simultaneously reducing their bioavailability. The essential features of the nutritional liquids and methods of making the nutritional liquids, as well as some of the many optional variations and additions, are described in detail hereafter.

The term “shelf stable” as used herein, unless otherwise specified, refers to a nutritional product that remains commercially stable after being packaged and then stored at 18-24° C. for at least 3 months, including from about 6 months to about 24 months, and also including from about 12 months to about 18 months.

The term “bioavailable” as used herein, unless otherwise specified, refers to the ability of a compound to enter into and remain in the bloodstream of an individual such that the substance can be absorbed into cells in the body. As the degree of bioavailability of a compound increases, the compound becomes more likely to enter into and remain in the bloodstream where it can be absorbed and used by the body. As the degree of bioavailability of a compound decreases, the compound becomes more likely to be expelled from the body before entering the bloodstream.

The term “nutritional liquid” as used herein, unless otherwise specified, refers to nutritional products in ready-to-drink liquid and concentrated liquid form.

The term “pediatric formula” as used herein, unless otherwise specified, refers to liquid human milk replacements or substitutes that are suitable for consumption by an infant or toddler up to the age of 36 months (3 years).

All percentages, parts and ratios as used herein, are by weight of the total liquid, unless otherwise specified. All such weights as they pertain to listed ingredients are based on the active level and, therefore, do not include solvents or by-products that may be included in commercially available materials, unless otherwise specified.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The various embodiments of the nutritional liquids of the present disclosure may also be substantially free of any optional or selected essential ingredient or feature described herein, provided that the remaining composition or liquid still contains all of the required ingredients or features as described herein. In this context, and unless otherwise specified, the term “substantially free” means that the selected liquid contains less than a functional amount of the optional ingredient, typically less than about 1%, including less than about 0.5%, including less than about 0.1%, and also including zero percent, by weight of such optional or selected essential ingredient.

The nutritional liquids may comprise, consist of, or consist essentially of the essential elements of the nutritional liquids as described herein, as well as any additional or optional element described herein or otherwise useful in nutritional liquid applications.

### Product Form

The present disclosure is directed to nutritional liquids that may be formulated with sufficient kinds and amounts of nutrients to provide a sole, primary, or supplemental source of nutrition, or to provide a specialized nutritional liquid for use in individuals afflicted with specific diseases or conditions or with a targeted nutritional benefit. The nutritional liquid including the metal amino acid chelate may be, for example, a preterm or term infant formula, a follow-on formula, a pediatric formula, a toddler formula, an adult nutritional formula, and the like.

The nutritional liquids include both concentrated and ready-to-feed nutritional liquids. Although the liquid form is not critical to the present disclosure, these nutritional liquids are most typically formulated as suspensions, emulsions or clear or substantially clear liquids.

Nutritional liquids in emulsion form suitable for use may be aqueous emulsions comprising proteins, fats, and/or carbohydrates. These emulsions are generally flowable or drinkable liquids at from about 1° C. to about 25° C. and are typically in the form of oil-in-water, water-in-oil, or complex aqueous emulsions, although such emulsions are most typically in the form of oil-in-water emulsions having a continuous aqueous phase and a discontinuous oil phase.

The nutritional liquids may be and typically are shelf stable. The nutritional liquids, typically contain up to about 95% by weight of water, including from about 50% to about 95%, also including from about 60% to about 90%, and also including from about 70% to about 85%, of water by weight of the nutritional liquid.

These nutritional liquids may have a variety of product densities, but most typically have a density greater than about 1.01 g/ml, including from about 1.06 g/ml to about 1.12 g/ml, and also including from about 1.085 g/ml to about 1.10 g/ml.

The nutritional liquid may have a pH ranging from about 2.5 to about 8, but are most advantageously in a range of from about 4.5 to about 7.5, including from about 5.5 to about 7.3, and including from about 6.2 to about 7.2.

### Metal Amino Acid Chelates

Nutritional liquids generally comprise vitamins or related nutrients, non-limiting examples of which include vitamin A, vitamin B12, vitamin C, and combinations thereof. As noted above, some of these nutrients may be decomposed by oxidation reactions in the nutritional liquids, potentially reducing the bioavailability of these nutrients and reducing the overall shelf life of the liquids.

The nutritional liquids of the present disclosure comprise metal amino acid chelates to minimize catalytic oxidation reactions in solution and the potential decomposition of oxidatively sensitive nutrients. Further, these metal amino acid chelates provide improved bioavailable forms of the metals (including iron and copper) in the chelates that may prove beneficial as additional nutritional components in the nutritional products. As such, the metal amino acid chelates can be used to fortify the nutritional liquids with needed metals, such as iron and copper, while simultaneously protecting sensitive nutrients from oxidation.

Non-limiting examples of suitable metal amino acid chelates for use in the nutritional liquids include copper lysine chelate, copper gluconate, copper bis-glycinate chelate, iron lysine chelate, iron gluconate, iron bis-glycinate chelate, and combinations thereof. In one particularly preferred embodiment, the metal amino acid chelate is copper lysine chelate. A commercially available source of a copper lysine chelate is CuPlex 100 (Zinpro Corporation, Edina Minn.). Other copper amino acid chelates are Instamin Copper (JH Biotch, Ventura, Calif.) and Copper Lysinate HCL Dihydrate (American International Chemical, Inc.). A commercially available source of copper bis-glycinate chelate is available from Albion Laboratories, Clearfield, Utah.

The concentration of metal amino acid chelates in the nutritional liquids may range from about 2 milligrams to about 3000 milligrams/liter of nutritional liquid, including from about 2 milligrams to about 2000 milligrams/liter of nutritional liquid, including from about 2 milligrams to about 400 milligrams/liter of nutritional liquid, including from about 4 milligrams to about 200 milligrams/liter of nutritional liquid, including from about 5 milligrams to about 150 milligrams/liter of nutritional liquid. In some embodiments, the concentration of metal amino acid chelates in the nutritional liquids may range from about 5 milligrams/liter to about 150 milligrams/liter of nutritional liquid, including from about 5 milligrams/liter to about 100 milligrams/liter, including from about 5 milligrams/liter to about 50 milligrams/liter of nutritional liquid.

Alternatively, in some embodiments, the concentration of metal amino acid chelates included in the nutritional liquid provides an equimolar concentration of the metal as is included in the nutritional liquid, typically as the corresponding metal sulfate; that is, the metal amino acid chelates provide an equimolar amount of the metal that is being replaced in the liquid nutritional. In one example, if a copper amino acid chelate is replacing a copper sulfate (pentahydrate), 1.85 grams of the copper amino acid chelate Copper Lysinate HCl Dihydrate is required to replace each 1.0 gram of copper sulfate (pentahydrate), as the copper amino acid chelate has a copper content of about 13.7% (w/w) and the copper sulfate (pentahydrate) has a copper content of about 25.4% (w/w).

### Macronutrients

The nutritional liquids may further comprise one or more optional macronutrients in addition to the metal amino acid chelates described herein. The optional macronutrients include proteins, lipids, carbohydrates, and combinations thereof. The nutritional liquids are desirably formulated as nutritional liquids containing all three macronutrients in addition to the metal amino acid chelates.

Macronutrients suitable for use herein include any protein, lipid, or carbohydrate or source thereof that is known for or otherwise suitable for use in an oral nutritional liquid, provided that the optional macronutrient is safe and effective for oral administration and is otherwise compatible with the other ingredients in the nutritional liquid.

The concentration or amount of optional lipid, carbohydrate, and protein in the nutritional liquid can vary considerably depending upon the particular nutritional application of the liquid (infant formulas, follow-on formulas, pediatric formulas, toddler formulas, adult nutritional formulas, etc.). These optional macronutrients are most typically formulated within any of the embodied ranges described in the following tables.

| Nutrient (% total calories) | Example A | Example B | Example C |
|---|---|---|---|
| Carbohydrate | 0-100 | 10-70 | 40-50 |
| Lipid | 0-100 | 20-65 | 35-55 |
| Protein | 0-100 | 5-40 | 15-25 |

Each numerical value preceded by the term “about”

| Nutrient (wt % composition) | Example D | Example E | Example F |
|---|---|---|---|
| Carbohydrate | 0-98 | 1-50 | 10-30 |
| Lipid | 0-98 | 1-30 | 3-15 |
| Protein | 0-98 | 1-30 | 2-10 |

Each numerical value preceded by the term “about”

### Carbohydrate

Optional carbohydrates suitable for use in the nutritional liquids may be simple, complex, or variations or combinations thereof, all of which are optionally in addition to the metal amino acid chelates as described herein. Non-limiting examples of suitable carbohydrates include hydrolyzed or modified starch or cornstarch, maltodextrin, isomaltulose, sucromalt, glucose polymers, sucrose, corn syrup, corn syrup solids, rice-derived carbohydrate, glucose, fructose, lactose, high fructose corn syrup, honey, sugar alcohols (e.g., maltitol, erythritol, sorbitol), and combinations thereof.

Optional carbohydrates suitable for use herein also include soluble dietary fiber, non-limiting examples of which include gum Arabic, fructooligosaccharide (FOS), sodium carboxymethyl cellulose, guar gum, citrus pectin, low and high methoxy pectin, oat and barley glucans, carrageenan, psyllium and combinations thereof. Insoluble dietary fiber is also suitable as a carbohydrate source herein, non-limiting examples of which include oat hull fiber, pea hull fiber, soy hull fiber, soy cotyledon fiber, sugar beet fiber, cellulose, corn bran, and combinations thereof.

In some embodiments, the carbohydrate concentration in the nutritional liquid may range from about 0.5% to about 50%, including from about 2% to about 30%, including from about 5% to about 25%, by weight of the nutritional liquid.

### Protein

Optional proteins suitable for use in the nutritional liquids in addition to the metal amino acid chelates include hydrolyzed, partially hydrolyzed or non-hydrolyzed proteins or protein sources, and can be derived from any known or otherwise suitable source such as milk (e.g., casein, whey), animal (e.g., meat, fish, egg albumen), cereal (e.g., rice, corn), vegetable (e.g., soy, pea, potato), or combinations thereof. The proteins for use herein can also include, or be entirely or partially replaced by, free amino acids known for use in nutritional products, non-limiting examples of which include L-tryptophan, L-glutamine, L-tyrosine, L-methionine, L-cysteine, taurine, L-arginine, L-carnitine, and combinations thereof.

In one embodiment, the protein source is a protein hydrolysate. In this context, the terms “protein hydrolysates” or “hydrolyzed protein” are used interchangeably herein and include extensively hydrolyzed proteins, wherein the degree of hydrolysis is most often at least about 20%, including from about 20% to about 80%, and also including from about 30% to about 80%, even more preferably from about 40% to about 60%. The degree of hydrolysis is the extent to which peptide bonds are broken by a hydrolysis method. The degree of protein hydrolysis for purposes of characterizing the extensively hydrolyzed protein component of these embodiments is easily determined by one of ordinary skill in the formulation arts by quantifying the amino nitrogen to total nitrogen ratio (AN/TN) of the protein component of the selected liquid formulation. The amino nitrogen component is quantified by USP titration methods for determining amino nitrogen content, while the total nitrogen component is determined by the Tecator Kjeldahl method, all of which are well known methods to one of ordinary skill in the analytical chemistry art.

Suitable protein hydrolysates may include soy protein hydrolysate, casein protein hydrolysate, whey protein hydrolysate, rice protein hydrolysate, potato protein hydrolysate, fish protein hydrolysate, egg albumen hydrolysate, gelatin protein hydrolysate, combinations of animal and vegetable protein hydrolysates, and combinations thereof Particularly preferred protein hydrolysates include whey protein hydrolysate and hydrolyzed sodium caseinate.

When used in the nutritional liquids, the protein source may include at least about 20% (by weight total protein) protein hydrolysate, including from about 30% to 100% (by weight total protein) protein hydrolysate, and including from about 40% to about 80% (by weight total protein) protein hydrolysate, and including about 50% (by weight total protein) protein hydrolysate. In one particular embodiment, the nutritional liquid includes 100% (by weight total protein) protein hydrolysate.

In some embodiments, the concentration of protein in the nutritional liquid may range from about 0.5% to about 30%, including from about 0.5% to about 20%, including from about 1% to about 20%, and also including from about 1.3% to about 10%, and also including from about 1% to about 7%, by weight of the nutritional liquid.

### Lipid

Optional lipids suitable for use in the nutritional liquids in addition to the metal amino acid chelates include coconut oil, fractionated coconut oil, soy oil, corn oil, olive oil, safflower oil, high oleic safflower oil, high GLA-safflower oil, MCT oil (medium chain triglycerides), sunflower oil, high oleic sunflower oil, palm and palm kernel oils, palm olein, canola oil, flaxseed oil, borage oil, cottonseed oils, evening primrose oil, blackcurrant seed oil, transgenic oil sources, fungal oils, marine oils (e.g., tuna, sardine) and so forth.

In some embodiments, the lipid may be present in the nutritional liquids in an amount of from 0% to about 30%, including from 1% to about 15% including from about 1% to about 10%, and including from about 2% to about 6% by weight of the nutritional liquid.

### Optional Ingredients

The nutritional liquids comprising the metal amino acid chelates may further comprise other optional ingredients that may modify the physical, nutritional, chemical, hedonic or processing characteristics of the products or serve as pharmaceutical or additional nutritional components when used in a targeted population. Many such optional ingredients are known or otherwise suitable for use in other nutritional liquids and may also be used in the nutritional liquids described herein, provided that such optional ingredients are safe and effective for oral administration and are compatible with the essential and other ingredients.

Non-limiting examples of such optional ingredients include preservatives, antioxidants, emulsifying agents, buffers, pharmaceutical actives, additional nutrients as described herein, colorants, flavors, thickening agents and stabilizers, and so forth.

In addition to vitamins A, B12, and C discussed above, the nutritional liquids may further comprise additional vitamins or related nutrients, non-limiting examples of which include vitamin D, vitamin E, vitamin K, thiamine, riboflavin, pyridoxine, carotenoids, niacin, folic acid, pantothenic acid, biotin, choline, inositol, salts, and derivatives thereof, and combinations thereof.

The nutritional liquids may further comprise additional minerals, non-limiting examples of which include phosphorus, magnesium, calcium, sodium, potassium, molybdenum, chromium, selenium, chloride, and combinations thereof.

The nutritional liquids may also include one or more flavoring or masking agents. Suitable flavoring or masking agents include natural and artificial sweeteners, sodium sources such as sodium chloride, and hydrocolloids, such as guar gum, xanthan gum, carrageenan, gellan gum, gum acacia and combinations thereof.

### Methods of Manufacture

The nutritional liquids may be manufactured by any known or otherwise suitable method for making nutritional liquids, including emulsions such as milk-based nutritional emulsions.

In one suitable manufacturing process, a nutritional liquid is prepared using at least three separate slurries, including a protein-in-fat (PIF) slurry, a carbohydrate-mineral (CHO-MIN) slurry, and a protein-in-water (PIW) slurry. The PIF slurry is formed by heating and mixing the selected oils (e.g., canola oil, corn oil, fish oil, etc.) and then adding an emulsifier (e.g., lecithin), fat soluble vitamins, and a portion of the total protein (e.g., milk protein concentrate, etc.) with continued heat and agitation. The CHO-MIN slurry is formed by adding with heated agitation to water: minerals (e.g., potassium citrate, dipotassium phosphate, sodium citrate, etc.), trace and ultra trace minerals (TM/UTM premix), thickening or suspending agents (e.g. Avicel, gellan, carrageenan), and metal amino acid chelates. The resulting CHO-MIN slurry is held for 10 minutes with continued heat and agitation before adding additional minerals (e.g., potassium chloride, magnesium carbonate, potassium iodide, etc.) and/or carbohydrates (e.g., fructooligosaccharide, sucrose, corn syrup, etc.). The PIW slurry is then formed by mixing with heat and agitation the remaining protein (e.g., sodium caseinate, soy protein concentrate, etc.) into water.

The resulting slurries are then blended together with heated agitation and the pH adjusted to the desired range, typically from 6.6-7.0, after which the composition is subjected to high-temperature short-time (HTST) processing during which the composition is heat treated, emulsified and homogenized, and then allowed to cool. Water soluble vitamins and ascorbic acid are added, the pH is again adjusted to the desired range if necessary, flavors are added, and water is added to achieve the desired total solid level. The composition is then aseptically packaged to form an aseptically packaged nutritional liquid, or the composition is added to retort stable containers and then subjected to retort sterilization to form retort sterilized nutritional liquids.

The manufacturing processes for the nutritional liquids may be carried out in ways other than those set forth herein without departing from the spirit and scope of the present disclosure. The present embodiments are, therefore, to be considered in all respects illustrative and not restrictive and that all changes and equivalents also come within the description of the present disclosure.

### Methods of Use

The use of metal amino acid chelates in the nutritional liquids provides an oxidatively shelf stable product having reduced off-color that is useful as a nutrition source, and may provide improved bioavailability of the metals contained in the amino acid chelates. Particularly, when used in a nutritional liquid with oxidatively sensitive nutrients such as vitamins A, B12 and C, the metal amino acid chelates decrease the ability of the incompletely bound metals to catalyze oxidation of these oxidation sensitive nutrients. For example, as the use of copper lysine chelate maintains copper in a completely bound form, copper lysine chelate in nutritional products shows decreases catalytic activity as compared to the respective copper sulfate salts typically used in nutritional liquids. This molecular form of copper (i.e., the copper lysine chelate) inhibits the capacity of the copper atom to catalyze the oxidation of nutrients, such as vitamin B12, vitamin A and vitamin C.

In some embodiments, the metal amino acid chelates are used in nutritional liquids that include cocoa powder. The color of nutritional liquids including cocoa powder may be negatively impacted by the metal complexation by the cocoa powder polyphenols. By including the metal amino acid chelates in the nutritional liquids including the cocoa powder, the metal complexation by the cocoa powder polyphenols can be minimized and the color of the liquid over time maintained.

In other embodiments, the metal amino acid chelates are used in nutritional liquids that include protein hydrolysates, including up to 100% protein hydrolysates (by weight total protein). Nutritional liquids that include extensively hydrolyzed proteins may be susceptible to oxidation of sensitive components. Accordingly, the use of metal amino acid chelates restricts catalytic metal interaction with vitamin C and vitamin B12, and other oxidatively sensitive nutrients.

### EXAMPLES

The following examples illustrate specific embodiments and or features of the nutritional liquids of the present disclosure. The examples are given solely for the purpose of illustration and are not to be construed as limitations of the present disclosure, as many variations thereof are possible without departing from the spirit and scope of the disclosure. All exemplified amounts are weight percentages based upon the total weight of the product, unless otherwise specified.

The exemplified products are nutritional liquids prepared in accordance with manufacturing methods well known in the nutrition industry for preparing nutritional emulsions.

### Example 1

In this Example, the capacity of copper lysine chelate to minimize vitamin B12 loss through oxidation in a nutritional emulsion is analyzed.

A commercially available chocolate flavored nutritional emulsion is used as the control emulsion and includes 35 μM of a copper ingredient as copper sulfate pentahydrate (CuSO₄5H₂O) and vitamin B12.

Three test emulsions are prepared according to Examples 5, 6, and 7 by substituting equimolar amounts of copper in the form of a copper amino acid chelate for the copper sulfate pentahydrate in the vitamin/mineral premix and retort sterilized. To provide equimolar amounts of copper, additional copper amino acid is included in the vitamin/mineral premix of the test emulsion samples as compared to the CuSO₄H₂O in the control sample. The emulsion samples and their respective copper components are shown in Table 1. The conversion factors, which provide grams of the various copper amino acid chelates needed to replace 1.0 gram of CuSO₄5H₂O are shown in Table 2.

Once prepared, the emulsions are analyzed for vitamin B12 recovery using a fully validated HPLC method to determine the amount of vitamin B12 remaining (i.e., not lost to oxidative destruction) in the nutritional emulsion. Further, the concentration of methionine sulfoxide, which is a marker of reactive oxygen species generation, is measured using the method disclosed in Baxter J H, et al., J. Chromatogr A, 1157 (2007) 10-16. The results are shown in Table 1.

TABLE 1

| | Methionine | | |
|---|---|---|---|
| | sulfoxide, as | | |
| | mole % of | | |
| Copper Ingredient | total | Vitamin B12 | Vitamin B12 |
| (35 μM) | methionine | (mcg/kg) | recovery v. control |
| Control (CuSO₄) | 18.8% | 38.5 | 100% |
| Copper bis-glycinate | 18.1% | 41.3 | 107% |
| chelate | | | |
| Copper gluconate | 17.9% | 46.4 | 121% |
| Copper lysine chelate | 17.4% | 47.0 | 122% |

TABLE 2

| | Conversion factor, as grams |
|---|---|
| | of copper amino acid chelate required |
| Copper Amino Acid Chelate | to replace 1.0 grams of CuSO₄•H₂0 |
| Copper lysine chelate (Copper | 1.85 |
| Lysinate HCl Dihydrate) | |
| Copper bis-glycinate chelate | 0.85 |
| Copper gluconate | 1.81 |

As shown in Table 1, including any of the copper amino acid chelates in the vitamin premix of the nutritional emulsion improves the stability of vitamin B12 as compared to the control emulsion, which included the conventional copper sulfate (pentahydrate). Copper lysine chelate and copper gluconate show the greatest abilities for preventing vitamin B12 decomposition, although all three copper amino acid chelates showed significant improvement as compared to the control.

### Example 2

In this Example, the ability of ferric salts and ferric amino acid chelates to minimize iron reactivity with cocoa powder is analyzed.

The visible absorbance of liquids including a mixture of cocoa powder and an iron-containing component is measured to determine the ability of various ferric salts, including ferric amino acid chelates, to decrease reactivity between the iron and the cocoa powder.

Various samples are prepared as described herein using various iron-containing components. All mixtures contain cocoa powder at 0.80% (w/w) in pH 6.8 buffer (0.10M HEPES). The control liquid is prepared without any iron-containing component. In the remaining samples, the iron components are added so that the weight ratio of cocoa powder:iron is 40:1 (w/w). All mixtures are then heated at 99° C. for thirty minutes and then filtered through a 0.45 μm membrane (Gelman Acrodisc #4497). The results are shown in Table 3, wherein lower numbers indicate less interaction with the cocoa powder and a performance that is closer to the iron-free control.

TABLE 3

| | Absorbance, | | |
|---|---|---|---|
| Iron-Containing | Filtrate v. Blank, mAU | | |
| Component | 535 nm* | 600 nm | 700 nm |
| Control (no iron) | 595 | 227 | 64 |
| Ferric orthophosphate | 579 | 237 | 65 |
| Ferric amino acid | 610 | 319 | 101 |
| chelate (Ferric bis- | | | |
| glycinate) | | | |
| Ferric pyrophosphate | 667 | 369 | 118 |
| Ferrous amino acid | 1200 | 1040 | 567 |
| chelate (Ferrous bis- | | | |
| glycinate) | | | |
| Ferrous gluconate | 1230 | 1100 | 607 |
| Ferrous sulfate | 1240 | 1150 | 688 |

*Iron chelates of tannic acid are known to absorb visibe light at 535 nm.

As shown in Table 3, the iron amino acid chelates (both ferric and ferrous) consistently exhibit lower reactivity (lower absorbance) with cocoa powder as compared to the ferrous sulfate. Specifically, the emulsion samples including the iron amino acid chelates performed more similarly to the iron-free control emulsion than did ferrous sulfate. Ferric orthophosphate and ferric pyrophosphate are poorly bioavailable (insoluble) iron salts. The benefits to using amino acid chelates is that there are both less reactive, but also equally or more bioavailable than the industry standard ferrous sulfate. These results show that the amino acid chelates are less reactive than ferrous sulfate, but at least equally or more bioavailable and better tolerated than ferrous sulfate.

### Example 3

In this Example, the ability of vitamin/mineral premixes including various ferric salts to minimize iron reactivity with cocoa powder in a nutritional emulsion is analyzed.

Specifically, the visible absorbance of cocoa powder and vitamin mineral premixes prepared with various ferric salts is compared to determine the ability of ferric salts, including ferric amino acid chelates, to prevent oxidation of the cocoa powder, which can lead to discoloration in nutritional liquids. Various samples are prepared using cocoa powder at 0.80% (w/w) in pH 6.8 buffer (0.10 HEPES). One control sample is prepared without any vitamin mineral premix or ferric salt. A second control sample is prepared with a vitamin premix, however, the vitamin premix does not include iron or copper. In the remaining samples, the ferric salts are added so that the weight ratio of cocoa powder:ferric salt is 220:1 (w/w). Approximately 0.0036% (w/w) vitamin mineral premix, including the ferric salts, is added to the cocoa powder. All mixtures are then heated at 99° C. for thirty minutes and then filtered through a 0.45 μm membrane (Gelman Acrodisc #4497). The results are shown in Table 4.

TABLE 4

| | Absorbance, Filtrate v. Blank, mAU | | | | |
|---|---|---|---|---|---|
| Premix/Ferric Salt | 535 nm* | 550 nm | 600 nm | 700 nm | 800 nm |
| Control 1 (none/none) | 582 | 452 | 226 | 102 | 86 |
| Control 2 (premix/no | 587 | 449 | 220 | 99 | 83 |
| iron, no copper) | | | | | |
| Premix/ferric | 591 | 452 | 219 | 97 | 80 |
| orthophosphate | | | | | |
| Premix/ferric | 594 | 454 | 221 | 99 | 84 |
| pyrophosphate | | | | | |
| Premix/ferric amino | 595 | 456 | 223 | 100 | 83 |
| acid chelate | | | | | |
| (Ferric bis-glycinate) | | | | | |
| Premix/ferrous sulfate | 599 | 471 | 249 | 111 | 93 |

*Iron chelates of tannic acid are known to absorb visible light at 535 nm.

As shown in Table 4, the addition of the vitamin mineral premix including ferrous sulfate shows the greatest absorption of visible light (i.e., the greatest emulsion discoloration) over the range tested. The amino acid chelates are preferred because they have less reactivity than the ferrous sulfate and are more bioavailable than the orthophosphate or pyrophosphate.

### Examples 4-8

Examples 4-8 illustrate nutritional emulsion embodiments of the present disclosure, the ingredients of which are listed in the table below. All amounts are listed as kilogram per 1000 kilogram batch of product, unless otherwise specified. The nutritional emulsions are prepared in accordance with the methods set forth herein.

| Ingredient | Ex. 4 | | Ex. 5 | | Ex. 6 | | Ex. 7 | | Ex. 8 | |
|---|---|---|---|---|---|---|---|---|---|---|
| Water | Q.S. | | Q.S. | | Q.S. | | Q.S. | | Q.S. | |
| Corn Syrup | 114.1 | | 114.1 | | 114.1 | | 114.1 | | 114.1 | |
| Sucrose | 53.6 | | 53.6 | | 53.6 | | 53.6 | | 53.6 | |
| Canola Oil | 26.2 | | 26.2 | | 26.2 | | 26.2 | | 26.2 | |
| Protein | 61.2 | | 61.2 | | 61.2 | | 61.2 | | 61.2 | |
| Corn Oil | 14.8 | | 14.8 | | 14.8 | | 14.8 | | 14.8 | |
| Cocoa Powder | 10.0 | | 10.0 | | 10.0 | | 10.0 | | 10.0 | |
| Fructooligosaccharides | 7.2 | | 7.2 | | 7.2 | | 7.2 | | 7.2 | |
| Potassium Citrate | 4.5 | | 4.5 | | 4.5 | | 4.5 | | 4.5 | |
| Lecithin | 1.6 | | 1.6 | | 1.6 | | 1.6 | | 1.6 | |
| Flavor Agents | 3.8 | | 3.8 | | 3.8 | | 3.8 | | 3.8 | |
| Stabilizer | 1.0 | | 1.0 | | 1.0 | | 1.0 | | 1.0 | |
| Magnesium Carbonate | 780.7 | g | 780.7 | g | 780.7 | g | 780.7 | g | 780.7 | g |
| Choline Chloride | 750.0 | g | 750.0 | g | 750.0 | g | 750.0 | g | 750.0 | g |
| Potassium Phosphate Dibasic | 336.8 | g | 336.8 | g | 336.8 | g | 336.8 | g | 336.8 | g |
| Potassium Chloride | 322.4 | g | 322.4 | g | 322.4 | g | 322.4 | g | 322.4 | g |
| Ascorbic Acid | 250.0 | g | 250.0 | g | 250.0 | g | 250.0 | g | 250.0 | g |
| UTM/TM Premix (Cu bis- | 242.0 | g | 0 | | 0 | | 0 | | 262.0 | g |
| glycinate chelate (7.06 g) replacing | | | | | | | | | | |
| copper sulfate pentahydrate on | | | | | | | | | | |
| equimolar basis) | | | | | | | | | | |
| UTM/TM Premix (Cu gluconate | 0 | | 243.0 | g | 0 | | 0 | | 0 | |
| (15.1 g) replacing copper sulfate | | | | | | | | | | |
| pentahydrate on equimolar basis) | | | | | | | | | | |
| UTM/TM Premix (Cu lysine | 0 | | 0 | | 243.0 | g | 174.4 | g | 0 | |
| chelate (15.4 g) replacing copper | | | | | | | | | | |
| sulfate pentahydrate on equimolar | | | | | | | | | | |
| basis) | | | | | | | | | | |
| Water Soluble Vitamin Premix | 242.0 | g | 242.0 | g | 242.0 | g | 242.0 | g | 242.0 | g |
| (including Vitamin B12) | | | | | | | | | | |
| L-Carnitine | 200.0 | g | 200.0 | g | 200.0 | g | 200.0 | g | 200.0 | g |
| 45% Potassium Hydroxide | 174.7 | g | 174.7 | g | 174.7 | g | 174.7 | g | 174.7 | g |
| Vitamin E | 39.0 | g | 39.0 | g | 39.0 | g | 39.0 | g | 39.0 | g |
| Suspending Agent | 25.0 | g | 25.0 | g | 25.0 | g | 25.0 | g | 25.0 | g |
| Beta Carotene | 14.0 | g | 14.0 | g | 14.0 | g | 14.0 | g | 14.0 | g |
| Vitamin A Palmitate in Corn Oil | 3.0 | g | 3.0 | g | 3.0 | g | 3.0 | g | 3.0 | g |
| Vitamin D3 in Corn Oil | 2.2 | g | 2.2 | g | 2.2 | g | 2.2 | g | 2.2 | g |
| Potassium Iodide | 194.0 | mg | 194.0 | mg | 194.0 | mg | 194.0 | mg | 194.0 | mg |

### Examples 9-12

Examples 9-12 illustrate pediatric nutritional emulsions of the present disclosure, the ingredients of which are listed in the table below. The nutritional emulsions are prepared in accordance with the methods set forth herein. All ingredient amounts are listed as kg per 1000 kg batch of product, unless otherwise specified.

| Ingredient | Ex. 9 | Ex. 10 | Ex. 11 | Ex. 12 |
|---|---|---|---|---|
| Water | Q.S. | Q.S. | Q.S. | Q.S. |
| Maltodextrin | 53 | 43.3 | 50 | 60 |
| Sucrose | 16.5 | 25 | 19.2 | 16.38 |
| Milk Protein Isolate | 15.65 | 15.65 | 15.65 | 15.65 |
| Corn Oil | 12 | 12 | 12 | 12 |
| High Oleic Safflower Oil | 10 | 10 | 10 | 10 |
| Monoglycerol Palmitate | 10 | 9 | 8 | 7 |
| Calcium Fatty Acid Salt | 6 | 7 | 8 | 9 |
| Coconut Oil | 2 | 2 | 2 | 2 |
| Fungal Oil | 0.3 | 0.3 | 0.3 | 0.3 |
| Lecithin | 0.1 | 0.1 | 0.1 | 0.1 |
| Potassium Phosphate | 0.96 | 0.96 | 0.96 | 0.96 |
| Dibasic | | | | |
| Potassium Chloride | 0.3 | 0.3 | 0.3 | 0.3 |
| Ascorbic Acid | 0.235 | 0.235 | 0.235 | 0.235 |
| Carrageenan | 0.150 | 0.150 | 0.150 | 0.150 |
| Potassium Hydroxide | 0.136 | 0.136 | 0.136 | 0.136 |
| UTM/TM Premix | 0.1684 | 0.1684 | 0.1684 | 0.1684 |
| (Copper Lysine Chelate; | | | | |
| Copper Lysinate HCl | | | | |
| Dihydrate as part premix) | | | | |
| Vitamin A, D, E Premix | 0.0758 | 0.0758 | 0.0758 | 0.0758 |
| Vitamin Premix | 0.0728 | 0.0728 | 0.0728 | 0.0728 |
| Potassium Iodide | 0.00022 | 0.00022 | 0.00022 | 0.00022 |
| Chromium Chloride | 0.000217 | 0.000217 | 0.000217 | 0.000217 |

### Examples 13-17

Examples 13-17 illustrate nutritional emulsions of the present disclosure, the ingredients of which are listed in the table below. The nutritional emulsions are prepared in accordance with the methods set forth herein. All ingredient amounts are listed as kg per 1000 kg batch of product, unless otherwise specified.

| Ingredient | Ex. 13 | Ex. 14 | Ex. 15 | Ex. 16 | Ex. 17 |
|---|---|---|---|---|---|
| Water | Q.S. | Q.S. | Q.S. | Q.S. | Q.S. |
| Maltodextrin | 99.2 | 99.2 | 99.2 | 99.2 | 99.2 |
| Whey Protein | 24.7 | 24.7 | 24.7 | 24.7 | 24.7 |
| Hydrolysate | | | | | |
| Sucrose | 21.9 | 21.9 | 21.9 | 21.9 | 21.9 |
| Canola oil | 16.6 | 16.6 | 16.6 | 16.6 | 16.6 |
| Medium-chain | 16.6 | 16.6 | 16.6 | 16.6 | 16.6 |
| triglycerides oil | | | | | |
| Hydrolyzed sodium | 12.6 | 12.6 | 12.6 | 12.6 | 12.6 |
| caseinate | | | | | |
| Whey protein | 5.1 | 5.1 | 5.1 | 5.1 | 5.1 |
| hydrolysate-lactalbumin | | | | | |
| hydrolysate | | | | | |
| Flavoring agent | 3.80 | 3.80 | 3.80 | 3.80 | 3.80 |
| Emulsifier | 1.8 | 1.8 | 1.8 | 1.8 | 1.8 |
| Precipitated calcium | 1.5 | 1.5 | 1.5 | 1.5 | 1.5 |
| carbonate | | | | | |
| Potassium chloride | 1.4 | 1.4 | 1.4 | 1.4 | 1.4 |
| Magnesium phosphate | 1.3 | 1.3 | 1.3 | 1.3 | 1.3 |
| Potassium citrate | 1.2 | 1.2 | 1.2 | 1.2 | 1.2 |
| Stabilizer | 1.0 | 1.0 | 1.0 | 1.0 | 1.0 |
| Sodium tripolyphosphate | 0.631 | 0.631 | 0.631 | 0.631 | 0.631 |
| Disodium phosphate | 0.610 | 0.610 | 0.610 | 0.610 | 0.610 |
| Choline chloride | 0.570 | 0.570 | 0.570 | 0.570 | 0.570 |
| UTM/TM premix | 0.365 | 0.3833 | 0.4015 | 0.347 | 0.329 |
| Copper lysine chelate; | 15 g | 11.5 g | 16.1 g | 10.5 g | 10.0 g |
| Copper Lysinate HCl | | | | | |
| Dihydrate (as part of | | | | | |
| UTM/TM premix) | | | | | |
| Liquid sucralose | 0.320 | 0.320 | 0.320 | 0.320 | 0.320 |
| Carrageenan | 0.300 | 0.300 | 0.300 | 0.300 | 0.300 |
| Sodium citrate | 0.242 | 0.242 | 0.242 | 0.242 | 0.242 |
| Ascorbic acid | 0.190 | 0.190 | 0.190 | 0.190 | 0.190 |
| Vitamin Premix | 0.160 | 0.160 | 0.160 | 0.160 | 0.160 |
| (FSMP premix) | | | | | |
| Potassium hydroxide | 0.133 | 0.133 | 0.133 | 0.133 | 0.133 |
| (45%) | | | | | |
| L-carnitine | 0.125 | 0.125 | 0.125 | 0.125 | 0.125 |
| Taurine | 0.110 | 0.110 | 0.110 | 0.110 | 0.110 |
| Vitamin A, D₃, E, | 0.025 | 0.025 | 0.025 | 0.025 | 0.025 |
| K₁ premix | | | | | |
| Potassium Iodide | 0.140 | 0.140 | 0.140 | 0.140 | 0.140 |

What is claimed is:

1. A nutritional liquid comprising from about 2 milligrams/liter to about 3000 milligrams/liter metal amino acid chelate and at least one vitamin.

2. The nutritional liquid of claim 1 comprising from about 2 milligrams/liter to about 400 milligrams/liter metal amino acid chelate.

3. The nutritional liquid of claim 1 comprising from about 4 milligrams/liter to about 200 milligrams/liter metal amino acid chelate.

4. The nutritional liquid of claim 1 comprising from about 5 milligrams/liter to about 150 milligrams/liter metal amino acid chelate.

5. The nutritional liquid of claim 1 wherein the metal amino acid chelate is selected from the group consisting of an iron amino acid chelate, a copper amino acid chelate, and combinations thereof.

6. The nutritional liquid of claim 5 wherein the metal amino acid chelate is a copper amino acid chelate selected from the group consisting of copper lysine chelates, copper gluconate, copper bis-glycinate chelate, and combinations thereof.

7. The nutritional liquid of claim 1 wherein the vitamin is selected from the group consisting of vitamin A, vitamin B12, vitamin C, and combinations thereof

8. The nutritional liquid of claim 1 comprising a protein hydrolysate.

9. The nutritional liquid of claim 1 further comprising cocoa powder.

10. The nutritional liquid of claim 1 wherein the nutritional liquid is selected from the group consisting of a preterm infant formula, a term infant formula, a follow-on formula, a pediatric formula, and a toddler formula.

11. A nutritional liquid comprising a protein hydrolysate, a metal amino acid chelate, and at least one vitamin.

12. The nutritional liquid of claim 11 wherein the protein hydrolysate is selected from the group consisting of a whey protein hydrolysate, a casein protein hydrolysate, and combinations thereof.

13. The nutritional liquid of claim 11 comprising 100% (by weight total protein) protein hydrolysate.

14. The nutritional liquid of claim 11 comprising from about 2 milligrams/liter to about 400 milligrams/liter metal amino acid chelate.

15. A nutritional liquid comprising from about 2 milligrams/liter to about 400 milligrams/liter copper amino acid chelate, cocoa powder, at least one of vitamin B12 or vitamin C, and at least one nutrient selected from the group consisting of a fat, a protein, or a carbohydrate.
